(19) United States
(12) Patent Application Publication
(10) Pub. No.: US 2025/0172541 A1
(43) Pub. Date: May 29, 2025

(54) IN VITRO BIOPRINTED BONE MODELS AND BIOINKS FOR BONE BIOPRINTING

(71) Applicant: Texas Tech University System, Lubbock, TX (US)

(72) Inventors: Sanjay K. Srivastava, Abilene, TX (US); Shreyas Gaikwad, Abilene, TX (US); Marina Curcic, Abilene, TX (US); Mohamed Eltokhy, Abilene, TX (US)

(73) Assignee: Texas Tech University System, Lubbock, TX (US)

(21) Appl. No.: 19/035,536

(22) Filed: Jan. 23, 2025

Related U.S. Application Data
(63) Continuation-in-part of application No. 18/126,214, filed on Mar. 24, 2023.
(60) Provisional application No. 63/323,156, filed on Mar. 24, 2022.

(51) Int. Cl. G01N 33/50; B33Y 10/00; B33Y 70/10; B33Y 80/00; C12N 5/071; C12N 5/077; C12N 5/09

(52) U.S. Cl. CPC G01N 33/5011; B33Y 10/00; B33Y 70/10; B33Y 80/00; C12N 5/0654; C12N 5/0693; C12N 5/0697; G01N 33/5082; C12N 2513/00; C12N 2533/54; C12N 2533/90

(57) ABSTRACT

Embodiments pertain to a bioink operational to form an artificial bone. The bioink includes a plurality of bone-related cells. Additional embodiments pertain to an artificial bone that includes a plurality of bone-related cells; and a bioink of the present disclosure that is embedded with the cells. The artificial bone may be in the form of a three-dimensional structure that includes a plurality of channels operational to allow fluid flow through the artificial bone. Further embodiments pertain to methods of making an artificial bone by applying a plurality of bone-related cells and a bioink onto a surface such that the bioink becomes embedded with the cells and forms a three-dimensional structure with a plurality of channels.

### CROSS-REFERENCE TO RELATED APPLICATIONS

This application is a continuation-in-part of U.S. patent application Ser. No. 18/126,214, filed on Mar. 24, 2023, which claims priority to U.S. Provisional Patent Application No. 63/323,156, filed on Mar. 24, 2022. The entirety of each of the aforementioned applications is incorporated herein by reference.

### BACKGROUND

Current in vitro bone models suffer from numerous limitations, such as a failure to resemble a bone's native environment. Numerous embodiments of the present disclosure aim to address the aforementioned limitations.

### SUMMARY

In some embodiments, the present disclosure pertains to a bioink operational to form an artificial bone. In some embodiments, the bioink includes a plurality of bone-related cells. In some embodiments, the plurality of bone-related cells include, without limitation, bone cancer cells, osteoblasts, osteoclasts, bone marrow cells, or combinations thereof. In some embodiments, the bioink further includes one or more components that include, without limitation, hydroxyapatite bone materials, fibrin, or combinations thereof.

Additional embodiments of the present disclosure pertain to an artificial bone that includes a plurality of bone-related cells; and a bioink of the present disclosure that is embedded with the cells. In some embodiments, the artificial bone mimics the structure of a bone. In some embodiments, the artificial bone is in the form of a three-dimensional structure that includes a plurality of channels. In some embodiments, the plurality of channels are operational to allow fluid flow through the artificial bone. In some embodiments, the plurality of channels also include at least one channel extending through an entire length of the artificial bone.

In some embodiments, the artificial bones of the present disclosure are associated with a bone maintenance system. In some embodiments, the bone maintenance system includes: a housing unit for housing the artificial bone; and a nutrient source in fluid communication with the housing unit. In some embodiments, the bone maintenance system further includes one or more additional housing units in fluid communication with the housing unit, where the one or more additional housing units contain one or more additional tissues.

Additional embodiments of the present disclosure pertain to methods of making an artificial bone by applying a plurality of bone-related cells of the present disclosure and a bioink of the present disclosure onto a surface. In some embodiments, the bioink becomes embedded with the cells. In some embodiments, the formed artificial bone is in the form of a three-dimensional structure with a plurality of channels.

In some embodiments, the formed artificial bone is utilized for evaluating one or more bone-related diseases. In some embodiments, the bone-related diseases include, without limitation, osteoarthritis, osteocarcinoma, bone-related cancers, bone cancer metastasis, or combinations thereof.

### DESCRIPTION OF THE DRAWINGS

FIG. 1A illustrates the architecture of an artificial bone in accordance with various embodiments of the present disclosure.

FIG. 1B illustrates a system for housing an artificial tissue.

FIG. 1C illustrates another system for housing an artificial bone.

FIG. 2A illustrates an extrusion bioprinting process for bioprinting an artificial tissue.

FIG. 2B shows images of in vitro printed tumor models.

FIGS. 3A-3D summarize experimental results related to bioink design and optimization.

FIGS. 4A-4C summarize experimental results related to cell proliferation in bioprinted tumors.

FIGS. 5A-5C show scanning electron microscopy (SEM) images of in-vivo pancreatic tumor tissues (FIG. 5A) and bio-printed tumors of AsPC-1 cells showing cell-matrix interactions (FIGS. 5B-5C).

FIGS. 6A-6H show anti-cancer efficacies of two-dimensional versus three-dimensional bio-printed pancreatic ductal adenocarcinoma (PDAC) tumor models after treatment with different anti-cancer drugs.

FIGS. 7A-7C show protein expression changes in two-dimensional and three-dimensional PDAC tumor models.

FIGS. 8A-8B illustrate the efficacy of bioprinted tumors for radiotherapy assessment.

FIGS. 9A-9C illustrate that bioprinted tumors can serve as a novel in vitro platform for immunotherapeutic analysis.

FIGS. 10A-10C further illustrate that bioprinted tumors can serve as a novel platform for immunotherapeutic analysis.

FIGS. 11A-11F show images of bioprinted blood vessels.

FIG. 12 shows an elastography of a bioprinted tumor.

FIG. 13 shows the cell viability of bone tumor cells on a scaffold.

FIGS. 14A-14B provide the results of cell viability assays, confirming that bone tumor cells proliferate.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose.

Currently, most in vitro studies of tissues, such as bones and tumors, are performed using two-dimensional assays. For instance, conventional in vitro techniques used to study anti-cancer activity of compounds consist of mainly two-dimensional techniques, where cells are layered on petri dishes. However, such two-dimensional environments do not mimic the in vivo tissue microenvironment. For instance, such models fail to recapitulate the physiology of human organs and tissues.

For instance, apart from the tumor core, a tumor's surrounding three-dimensional extra cellular matrix (ECM) plays a crucial role in tumor progression. The ECM expands as the tumor progresses, thus changing the mechanical characteristics of the whole tissue as well as the individual cells.

Moreover, current bioinks used to make tissue models suffer from numerous limitations. For instance, many existing bioinks include single agents derived from animal or plant sources.

As such, a need exists to develop improved tissue models and bioinks to generate such tissue models, such as bone models and tumor models. Numerous embodiments of the present disclosure aim to address the aforementioned need.

### Bioink Compositions

In some embodiments, the present disclosure pertains to a bioink composition. In some embodiments, the bioink is operational to form an artificial tissue. In some embodiments, the bioink is operational to form an artificial bone.

In some embodiments, the bioink mimics an extra-cellular matrix (ECM) composition of a tissue, such as a tumor, a bone, or combinations thereof. In some embodiments, the bioink mimics the extra-cellular matrix (ECM) composition of pancreatic ductal adenocarcinoma (PDAC) tumors. In some embodiments, the bioink mimics the extra-cellular matrix (ECM) composition of a bone.

In some embodiments, the bioinks of the present disclosure include materials derived completely from humans. In some embodiments, the bioink consists of materials derived completely from humans. In some embodiments, the bioinks of the present disclosure include materials derived completely from human tissues.

In some embodiments, the majority of the bioink is derived from animals. In some embodiments, at least 50% by weight of the bioink is derived from animals. In some embodiments, at least 60% by weight of the bioink is derived from animals. In some embodiments, at least 70% by weight of the bioink is derived from animals. In some embodiments, at least 80% by weight of the bioink is derived from animals. In some embodiments, at least 85% by weight of the bioink is derived from animals. In some embodiments, at least 90% by weight of the bioink is derived from animals. In some embodiments, at least 95% by weight of the bioink is derived from animals. In some embodiments, at least 99% by weight of the bioink is derived from animals.

In some embodiments, the majority of the bioink is derived from humans. In some embodiments, at least 50% by weight of the bioink is derived from humans. In some embodiments, at least 60% by weight of the bioink is derived from humans. In some embodiments, at least 70% by weight of the bioink is derived from humans. In some embodiments, at least 80% by weight of the bioink is derived from humans. In some embodiments, at least 85% by weight of the bioink is derived from humans. In some embodiments, at least 90% by weight of the bioink is derived from humans. In some embodiments, at least 95% by weight of the bioink is derived from humans. In some embodiments, at least 99% by weight of the bioink is derived from humans.

In some embodiments, the bioinks of the present disclosure include less than 40% by weight of non-human animal-derived materials. In some embodiments, the bioinks of the present disclosure include less than 20% by weight of non-human animal-derived materials. In some embodiments, the bioinks of the present disclosure include less than 10% by weight of non-human animal-derived materials. In some embodiments, the bioinks of the present disclosure include less than 5% by weight of non-human animal-derived materials. In some embodiments, the bioinks of the present disclosure include less than 1% by weight of non-human animal-derived materials.

In some embodiments, the bioinks of the present disclosure include hydrogels. In some embodiments, the bioinks of the present disclosure include collagens. In some embodiments, the collagens are at concentrations that mimic collagen concentrations in human tissues, bones or tumors. For instance, in some embodiments, the bioinks of the present disclosure include at least 10% by weight of collagen. In some embodiments, the bioinks of the present disclosure include at least 15% by weight of collagen. In some embodiments, the bioinks of the present disclosure include at least 20% by weight of collagen. In some embodiments, the bioinks of the present disclosure include at least 30% by weight of collagen. In some embodiments, the bioinks of the present disclosure include at least 40% by weight of collagen. In some embodiments, the bioinks of the present disclosure include at least 50% by weight of collagen. In some embodiments, the bioinks of the present disclosure include at least 60% by weight of collagen. In some embodiments, the bioinks of the present disclosure include at least 70% by weight of collagen. In some embodiments, the bioinks of the present disclosure include at least 80% by weight of collagen. In some embodiments, the bioinks of the present disclosure include at least 85% by weight of collagen.

In some embodiments, the collagen in the bioinks of the present disclosure include collagen I, collagen III, and collagen IV. In some embodiments, the collagen I, collagen III, and collagen IV are derived from humans.

In some embodiments, the bioinks of the present disclosure include at least 25% by weight of collagen I. In some embodiments, the bioinks of the present disclosure include at least 40% by weight of collagen I. In some embodiments, the bioinks of the present disclosure include at least 50% by weight of collagen I. In some embodiments, the bioinks of the present disclosure include at least 60% by weight of collagen I.

In some embodiments, the bioinks of the present disclosure include at least 5% by weight of collagen III. In some embodiments, the bioinks of the present disclosure include at least 7.5% by weight of collagen III. In some embodiments, the bioinks of the present disclosure include at least 10% by weight of collagen III. In some embodiments, the bioinks of the present disclosure include at least 15% by weight of collagen III.

In some embodiments, the bioinks of the present disclosure include at least 1% by weight of collagen IV. In some embodiments, the bioinks of the present disclosure include at least 2.5% by weight of collagen IV. In some embodiments, the bioinks of the present disclosure include at least 5% by weight of collagen IV.

In some embodiments, the bioinks of the present disclosure include at least 25% by weight of collagen I, at least 5% by weight of collagen III, and at least 1% by weight of collagen IV. In some embodiments, the bioinks of the present disclosure include at least 60% by weight of collagen I, at least 15% by weight of collagen III, and at least 5% by weight of collagen IV.

The bioinks of the present disclosure may include one or more additional components. For instance, in some embodiments, the bioinks of the present disclosure may include without limitation, sodium alginate, gelatin, fibronectin, laminin, hydroxyapatite bone materials, fibrin, poly-caprolactone, osteocalcin, osteonectin, ostopontin, or combinations thereof. In some embodiments, the bioinks of the present disclosure may include, without limitation, hydroxyapatite bone materials, fibrin, or combinations thereof.

In some embodiments, the bioinks of the present disclosure include at least 1% by weight of gelatin. In some embodiments, the bioinks of the present disclosure include at least 5% by weight of gelatin. In some embodiments, the bioinks of the present disclosure include at least 7.5% by weight of gelatin. In some embodiments, the bioinks of the present disclosure include at least 10% by weight of gelatin.

In some embodiments, the bioinks of the present disclosure include at least 1% by weight of sodium alginate. In some embodiments, the bioinks of the present disclosure include at least 5% by weight of sodium alginate. In some embodiments, the bioinks of the present disclosure include at least 7.5% by weight of sodium alginate. In some embodiments, the bioinks of the present disclosure include at least 10% by weight of sodium alginate. In some embodiments, the bioinks of the present disclosure include at least 60% by weight of collagen I, at least 15% by weight of collagen III, at least 5% by weight of collagen IV, at least 10% by weight of gelatin, and at least 10% by weight of sodium alginate.

In some embodiments, the bioinks of the present disclosure may also include cells. For instance, in some embodiments, the cells include tissue cells, tumor cells, cancer cells, immune cells, fibroblasts, or combinations thereof.

In some embodiments, the bioinks of the present disclosure may also include bone-related cells. In some embodiments, the bone-related cells include, without limitation, bone tissue cells, lung tissue, tumor cells, cancer cells, bone cancer cells, immune cells, fibroblasts, osteoblasts, osteoclasts, bone marrow cells, or combinations thereof. In some embodiments, the bone-related cells include, without limitation, bone cancer cells, osteoblasts, osteoclasts, bone marrow cells, or combinations thereof.

In some embodiments, the bioinks of the present disclosure include tissue cells. In some embodiments, the bioinks of the present disclosure include tumor cells. In some embodiments, the bioinks of the present disclosure include cancer cells. In some embodiments, the cancer cells include, without limitation, pancreatic cancer cells, pancreatic ductal adenocarcinoma cells, brain cancer cells, breast cancer cells, lung cancer cells, colon cancer cells, renal cancer cells, gastrointestinal cancer cells, sarcoma cells, bone cancer cells, or combinations thereof. In some embodiments, the cancer cells include pancreatic ductal adenocarcinoma cells.

In some embodiments, the bioinks of the present disclosure include fibroblasts. In some embodiments, the bioinks of the present disclosure include immune cells. In some embodiments, the immune cells include, without limitation, peripheral blood mononuclear cells (PBMCs), lymphocytes, monocytes, natural killer cells (NK cells), dendritic cells, T-cells, B-cells, endothelial cells, bone marrow derived cells, cancer associated fibroblasts, or combinations thereof.

In some embodiments, the cells include a biomarker for imaging the cells. In some embodiments, the biomarker includes, without limitation, fluorescent proteins, green fluorescent proteins, luciferase, or combinations thereof.

### Artificial Tissues

Additional embodiments of the present disclosure pertain to an artificial tissue. In some embodiments, the artificial tissue includes a plurality of cells and a bioink of the present disclosure. In some embodiments, the bioink is embedded with the cells. In some embodiments, the artificial tissue is in the form of a three-dimensional structure. In some embodiments, the three-dimensional structure includes a plurality of channels.

### Artificial Tissue Forms

The artificial tissues of the present disclosure may be in various forms. For instance, in some embodiments, the artificial tissues of the present disclosure are in the form of an extracellular matrix. In some embodiments, the artificial tissues of the present disclosure are in the form of a blood vessel. In some embodiments, the artificial tissues of the present disclosure are in the form of a tumor.

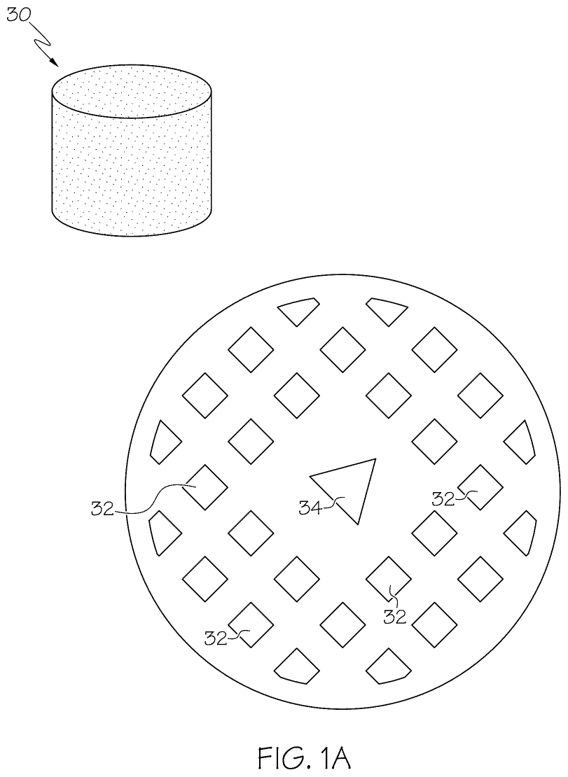

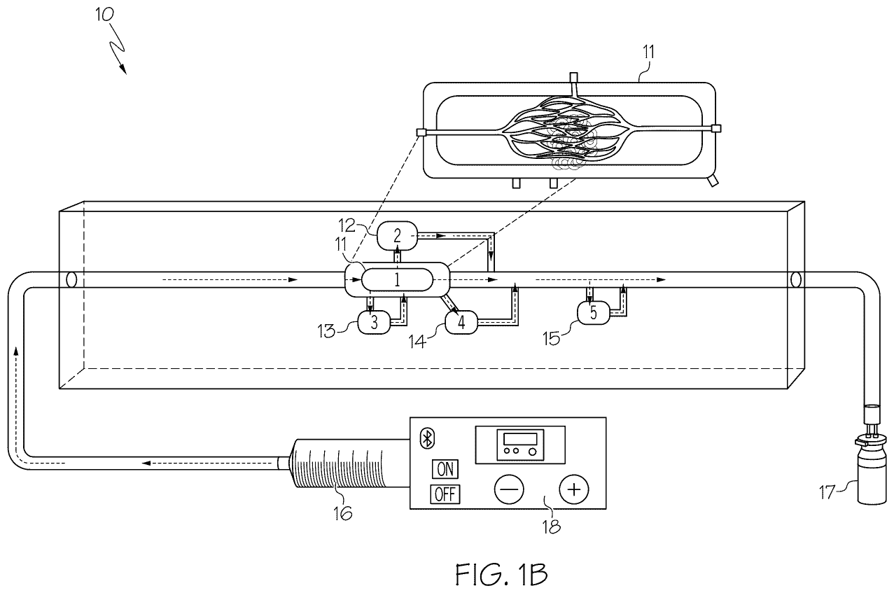

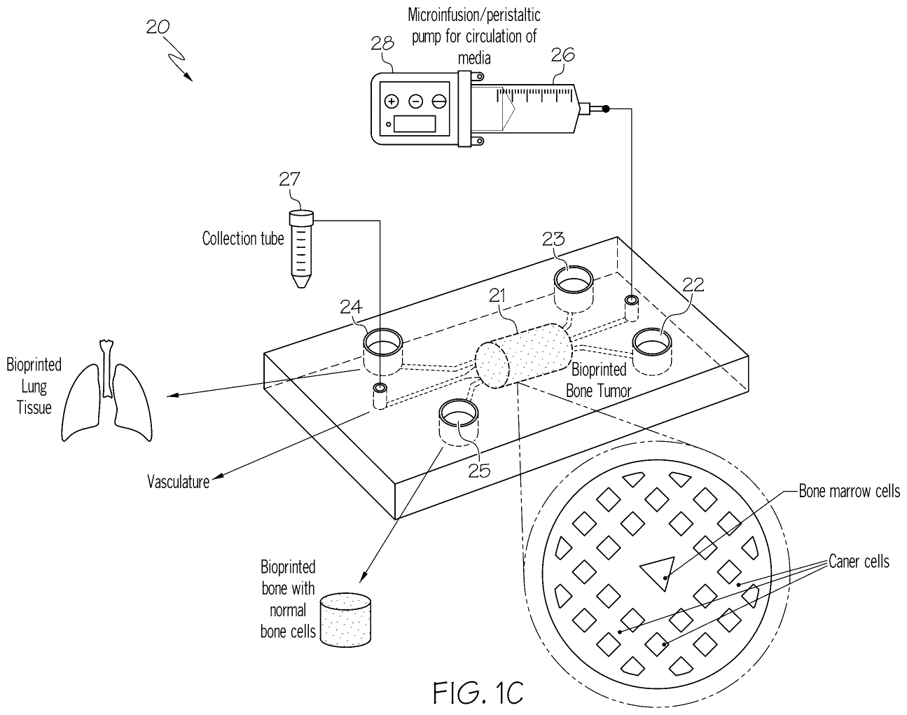

In some embodiments illustrated in FIG. 1A, the artificial tissues of the present disclosure are in the form of an artificial bone 30. In some embodiments, artificial bone 30 includes: a plurality of bone-related cells; and a bioink embedded with the cells. In some embodiments, the bioink includes at least 20% by weight of collagen. In some embodiments, artificial bone 30 is in the form of a three-dimensional structure with a plurality of channels 32. In some embodiments, the plurality of channels 32 are operational to allow fluid flow through the artificial bone. In some embodiments, the plurality of channels include at least one channel 34 extending through an entire length of the artificial bone. In some embodiments, such a channel 34 is positioned at a central region of artificial bone 30.

In some embodiments, the artificial tissues of the present disclosure are in the form of a tumor micro-environment. In some embodiments, the artificial tissues of the present disclosure mimic the tumor micro-environment of a cancer. In some embodiments, the cancer includes, without limitation, pancreatic cancer, pancreatic ductal adenocarcinoma, brain cancer, breast cancer, lung cancer, colon cancer, renal cancer, gastrointestinal cancer, sarcoma, bone cancer, or combinations thereof. In some embodiments, the cancer is pancreatic ductal adenocarcinoma (PDAC).

In some embodiments, the artificial tissues of the present disclosure are in the form of a bone cancer model. In some embodiments, the artificial bone is in the form of a bone micro-environment.

### Cells

The artificial tissues of the present disclosure may include various cells. For instance, in some embodiments, the cells include tumor cells. In some embodiments, the cells include tissue cells. In some embodiments, the cells include cancer cells. In some embodiments, the cancer cells include, without limitation, pancreatic cancer cells, pancreatic ductal adenocarcinoma cells, brain cancer cells, breast cancer cells, lung cancer cells, colon cancer cells, renal cancer cells, gastrointestinal cancer cells, sarcoma cells, bone cancer cells, or combinations thereof. In some embodiments, the cancer cells include pancreatic ductal adenocarcinoma cells (PDAC).

In some embodiments, the artificial tissues of the present disclosure include immune cells. In some embodiments, the immune cells include, without limitation, peripheral blood mononuclear cells (PBMCs), lymphocytes, monocytes, natural killer cells (NK cells), dendritic cells, T-cells, B-cells, endothelial cells, bone marrow derived cells, cancer associated fibroblasts, or combinations thereof. In some embodiments, the artificial tissues of the present disclosure include fibroblasts.

In some embodiments, the artificial tissues of the present disclosure may include bone-related cells. In some embodiments, the bone-related cells include, without limitation, bone tissue cells, lung tissue, tumor cells, cancer cells, bone cancer cells, immune cells, fibroblasts, osteoblasts, osteoclasts, bone marrow cells, or combinations thereof. In some embodiments, the bone-related cells include, without limitation, bone cancer cells, osteoblasts, osteoclasts, bone marrow cells, or combinations thereof.

In some embodiments, the artificial tissues of the present disclosure include cells with a biomarker for imaging the cells. In some embodiments, the biomarker includes, without limitation, fluorescent proteins, green fluorescent proteins, luciferase, or combinations thereof.

### Housing Units

In some embodiments, the artificial tissues of the present disclosure may be associated with a housing unit for storing and maintaining the artificial tissue. In some embodiments, the housing unit includes, without limitation, a petri dish, a chip, a tissue culture system, a tissue maintenance system, or combinations thereof.

In some embodiments, the housing unit is in the form of a tissue maintenance system. In some embodiments, the tissue maintenance system includes: a housing unit for housing the artificial tissue; and a nutrient source in fluid communication with the housing unit. In some embodiments, the system also includes one or more additional housing units in fluid communication with the housing unit. In some embodiments, the one or more additional housing units contain one or more additional tissues.

An example of a tissue maintenance system is illustrated in FIG. 1B as system 10. In this example, system 10 includes housing unit 11 for housing an artificial tissue; and a nutrient source 16 in fluid communication with housing unit 11. In this example, system 10 also includes additional housing units 12, 13, 14, and 15 that are each in fluid communication with housing unit 11. The additional housing units may contain one or more additional tissues, such as liver tissue (e.g., in housing unit 12), lymph node tissue (e.g., in housing unit 13), lung tissue (e.g., in housing unit 14), and other tissues of interest for mimicking a tissue environment, such as a tumor microenvironment (e.g., in housing unit 15).

System 10 also includes a fluid pump 18 (e.g., a Blue tooth enabled fluid pump) for pumping nutrients from nutrient source 16 through the system 10. Additionally, system 10 includes collection tube 17 for collecting nutrients and other byproducts from the system. In some embodiments, collection tube 17 can be utilized to collect media and various biomarkers produced by cells, such as cytokines, circulating tumor cells, chemicals produced by cells, drugs, compounds, or combinations thereof. In some embodiments, the collected materials are utilized for research and development purposes.

In some embodiments, fluid pump 18 is in the form of a Bluetooth enabled fluid pump. In some embodiments, the Bluetooth enabled fluid pump reduces a need for complex pumping systems.

Another example of a tissue maintenance system is illustrated in FIG. 1C as bone maintenance system 20. In this example, bone maintenance system 20 includes housing unit 21 for housing an artificial bone; and a nutrient source 26 in fluid communication with housing unit 21. In this example, system 20 also includes additional housing units 22, 23, 24, and 25 that are each in fluid communication with housing unit 21. The additional housing units may contain one or more additional tissues, such as liver tissue (e.g., in housing unit 22), lymph node tissue (e.g., in housing unit 23), lung tissue (e.g., in housing unit 24), and other tissues of interest for mimicking a bone environment, such as a bone tumor microenvironment (e.g., in housing unit 25).

System 20 also includes a fluid pump 28 (e.g., a Blue tooth enabled fluid pump) for pumping nutrients from nutrient source 26 through the system. Additionally, system 20 includes collection tube 27 for collecting nutrients and other byproducts from the system. In some embodiments, collection tube 27 can be utilized to collect media and various biomarkers produced by bone cells, such as cytokines, circulating tumor cells, chemicals produced by cells, drugs, compounds, or combinations thereof. In some embodiments, the collected materials are utilized for research and development purposes.

In some embodiments, fluid pump 28 is in the form of a Bluetooth enabled fluid pump. In some embodiments, the Bluetooth enabled fluid pump reduces a need for complex pumping systems.

### Methods of Making Artificial Tissues

Additional embodiments of the present disclosure pertain to methods of making an artificial tissue, such as the artificial tissues of the present disclosure (e.g., artificial bones). In some embodiments, the methods of the present disclosure include applying a plurality of cells of the present disclosure and a bioink of the present disclosure onto a surface. In some embodiments, the bioink becomes embedded with the cells. In some embodiments, the formed artificial tissue is in the form of a three-dimensional structure. In some embodiments, the three-dimensional structure includes a plurality of channels.

In some embodiments, the methods of the present disclosure also include a step of mixing the bioinks of the present disclosure with the cells. In some embodiments, the mixing step occurs prior to the applying step. In some embodiments, the mixing step occurs during the applying step.

In some embodiments, the methods of the present disclosure also include a step of mixing the bioinks and cells of the present disclosure with one or more cross-linking agents. In some embodiments, the mixing step occurs prior to the applying step. In some embodiments, the mixing step occurs during the applying step.

Various methods may be utilized to apply bioinks and cells onto a surface. For instance, in some embodiments, the applying occurs by three-dimensional printing (also referred to as bioprinting). In some embodiments, the three-dimensional printing includes extrusion bioprinting. In some embodiments, extrusion bioprinting enables extrusion of a mixture of bioink and cells using a positive pressure pump and a robotic arm of a three-dimensional printer. In some embodiments, three-dimensional printing includes fresh bioprinting.

In some embodiments, the bioink and the bone-related cells are applied onto a surface at the same time. In some embodiments, the bioink and the bone-related cells are applied onto the surface sequentially. For instance, in some embodiments, the method includes: applying the bioink onto the surface to form a three-dimensional structure with a plurality of channels; and applying the bone-related cells onto the three-dimensional structure. In some embodiments, the bone-related cells are applied into the plurality of channels of the three-dimensional structure.

### Applications

The artificial tissues of the present disclosure can have various applications. For instance, in some embodiments, the artificial tissues may be utilized for screening therapeutics. In some embodiments, the therapeutics include, without limitation, radiation therapy, chemotherapy, immunotherapy, drugs, drug candidates, or combinations thereof.

In some embodiments, the artificial bones of the present disclosure may be utilized for evaluating one or more bone-related diseases. In some embodiments, the bone-related diseases include, without limitation, osteoarthritis, osteocarcinoma, bone-related cancers, bone cancer metastasis, or combinations thereof.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Bioprinted Pancreatic Tumor: A Novel Model Recapitulating the Tumor Microenvironment

Pancreatic ductal adenocarcinoma (PDAC) has a complex tumor microenvironment (TME) with multiple factors governing the tumor progression. Factors such as the mechanical stiffness of the tumor play an important role, especially in solid tumors like PDAC. The presence or absence of multiple cell types such as cancer-associated fibroblasts and immune cells also play a critical role in tumor growth and response to chemotherapies. Conventional techniques use two-dimensional techniques which fail to mimic the complex PDAC TME.

In this Example, Applicant developed the first-ever bioprinted model of PDAC using extrusion bioprinting. Applicant's model closely mimics some important hallmarks of PDAC. In particular, Applicant developed a specialized bioink that closely mimics the stromal desmoplasia of the TME. The bioink is completely derived from human tissues, thereby making it a true replica of the actual in vivo environment.

Applicant bioprinted various tumor models using several human PDAC cell lines. Applicant established the effect of bioink on the post-print viability and monitored the proliferation of PDAC cells. Applicant characterized the tumor model as a platform for chemotherapeutic drug screening, radiotherapeutic screening and established it as a platform for immune activity studies. In the current study, through extensive characterization, Applicant has established a first-of-its-kind bioprinted PDAC model using a novel bioink.

In particular, the bioink in this Example is the only bioink that closely mimics the human PDAC tumor environment. Applicant's bioink contains less percentage of animal derived tissue material as compared to others. The bioink contains collagen I, collagen III and collagen IV. The content of collagen I is the highest, which is the case of any PDAC tumor microenvironment. Moreover, the specific ratio of these different collagens and other material is unique to this invention.

### Example 1.1. Experimental Methods

An exemplary bioink is complete and has been optimized for bioprinting using a bioprinter (Allevi 3D).

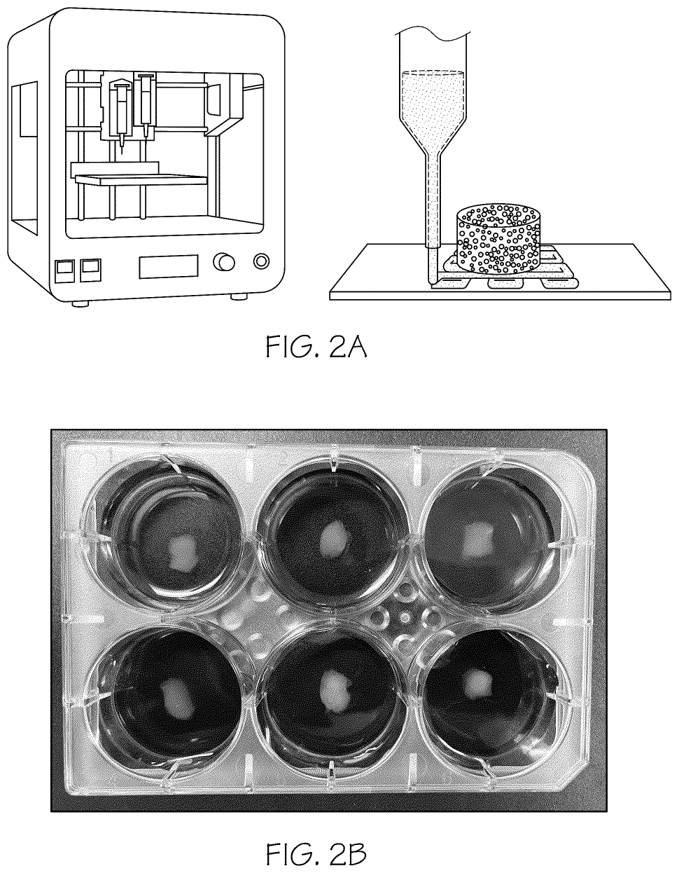

As illustrated in FIGS. 2A-2B, the bioprinting of PDAC tumors was performed using the Allevi 3 Bioprinter (3D Systems Ltd). PDAC cells were collected from their respective culture mediums and mixed with an in house bioink. This was followed by addition of the bioink-cell mixture to a 5 mL cartridge. Upon attainment of the specified nozzle temperature, printing instructions were sent from the Allevi Software. Post printing, the tumors were submerged in crosslinking solution for 1-2 minutes and then washed twice with PBS. The tumors were then placed in respective media and used for further studies.

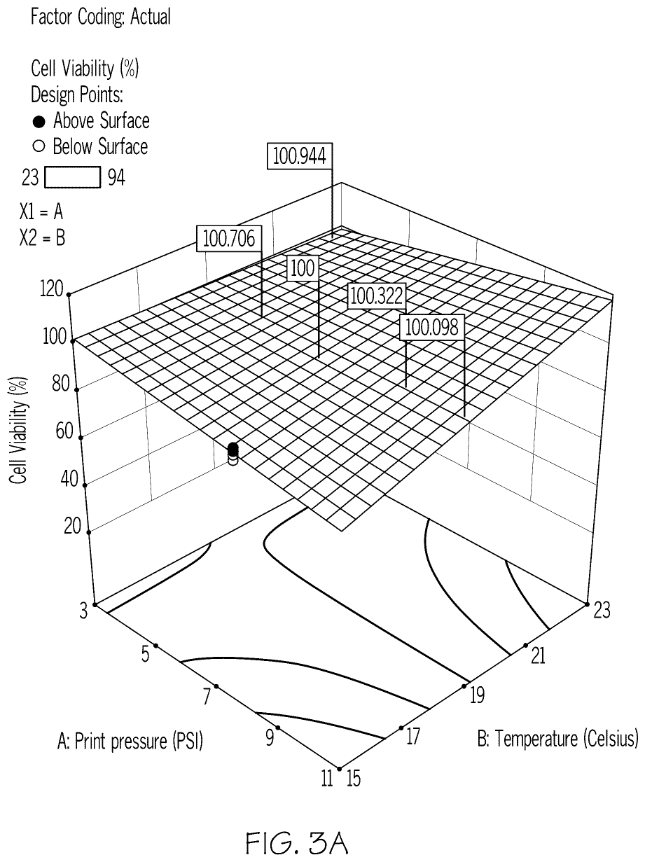

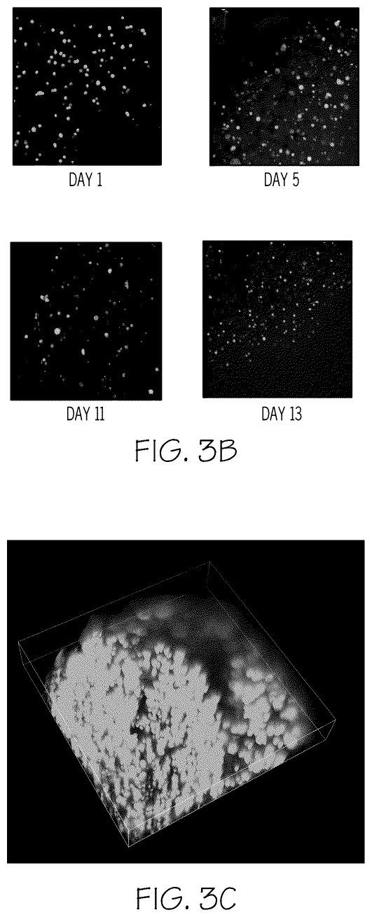

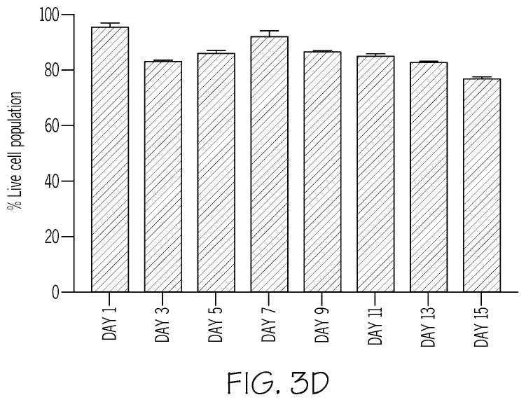

As illustrated in FIGS. 3A-3C, the in-house bioink was optimized using a design of experiments (DOE) approach, where temperature and print pressure were used as variables and cell viability post printing was considered as the output.

Cell viability was analyzed using calcein AM (for imaging live cells since calcein AM turns Green when inside live cells due to the enzymatic activity in the cells) and ethidium homodimer (for imaging dead cells).

FIG. 3A shows the DOE optimization design. FIG. 3B shows the live/dead imaging using a Nikon Confocal Microscope. FIG. 3C shows an additional image captured using the Nikon Confocal Microscope in a Z-stack manner. Z-stack capturing allowed Applicant to capture the cells present in all the layers of the bioprinted tumors and confirm that cells present across the whole tumor are live and functional for conducting further experiments. FIG. 3D shows cell viability (representative data for n=3) values that were plotted as means ±SD.

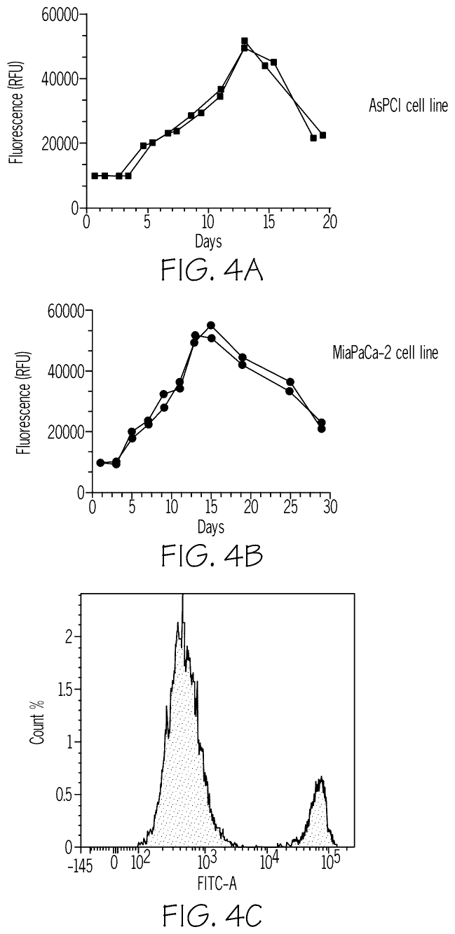

As illustrated in FIGS. 4A-4C, Applicant also performed cell proliferation analyses. The proliferation of bioprinted tumors was monitored using Alamar Blue dye from day 1 to day 30. FIG. 4A shows cell proliferation for AsPC1. FIG. 4B shows cell proliferation curve for MiaPaCa-2 cells. FIG. 4C shows CFSE analysis to monitor cell proliferation in order to support the findings from the Alamar Blue assay.

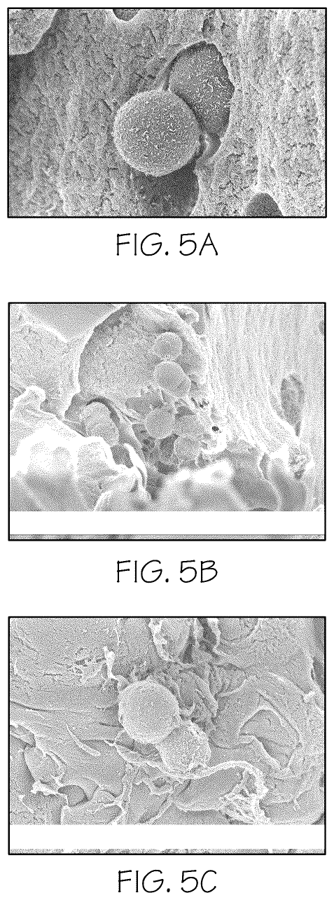

FIG. 5A shows in-vivo pancreatic tumor tissues. FIGS. 5B-5C show bioprinted tumors of AsPC-1 cells showing cell-matrix interactions. Imaging was performed on Hitachi S-4300 microscopes.

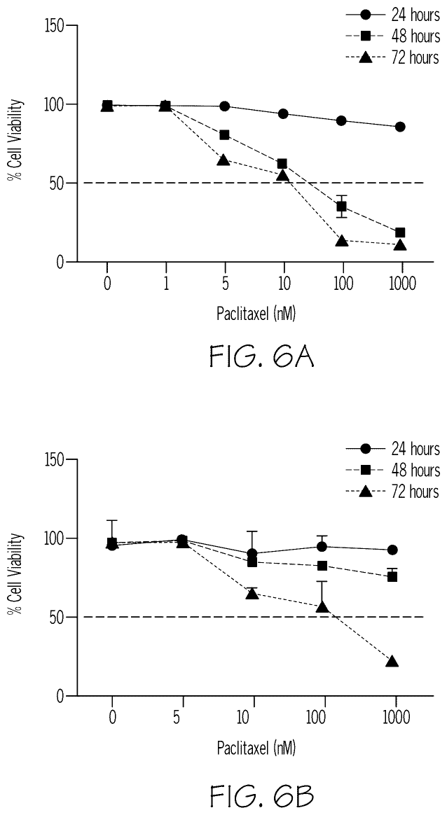

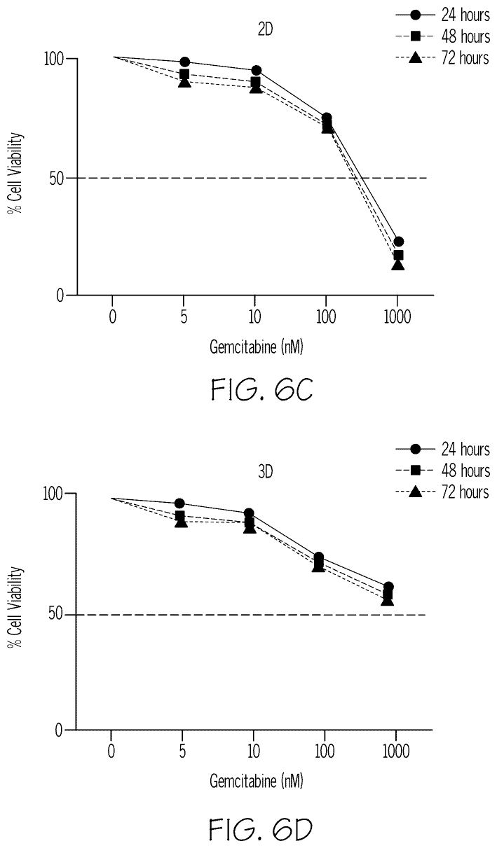

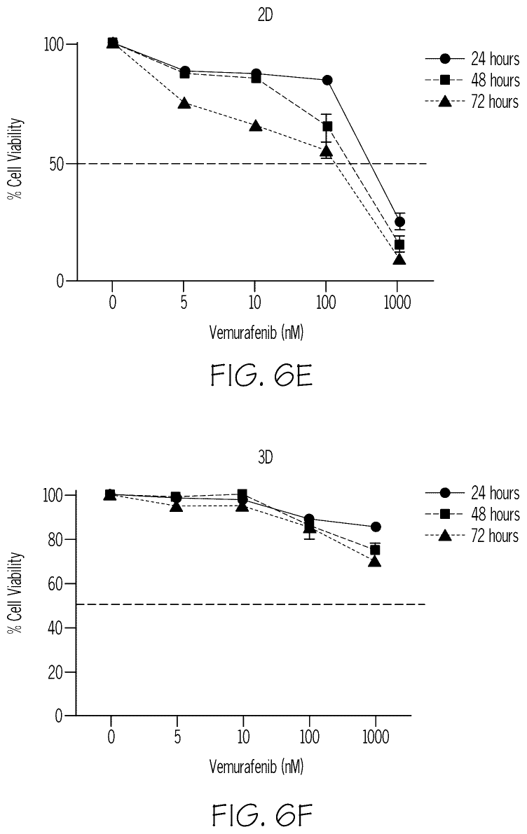

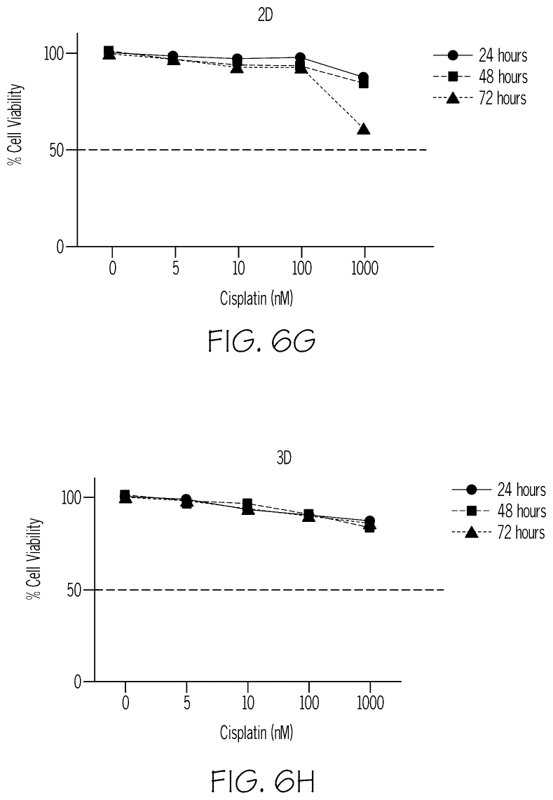

As shown in FIGS. 6A-6H, the anti-cancer efficacies of different chemotherapeutic drugs were studied in two-dimensional cell cultures and three-dimensional bioprinted tumors using ATP Cell Titer Glo assay kits (Promega Ltd.). The assay is based on the measurement of ATP levels, which are a direct indicator of cellular viability.

In particular, the cytotoxicity of Paclitaxel in two-dimensional culture (FIG. 6A) and bioprinted tumors (FIG. 6B) were compared. Values were plotted as means ±SD. Similarly, the cytotoxicity of Gemcitabine, Vemurafenib, and Cisplatin in two-dimensional culture (FIGS. 6C, 6E and 6G) and bioprinted tumors (FIGS. 6D, 6F and 6H) were compared.

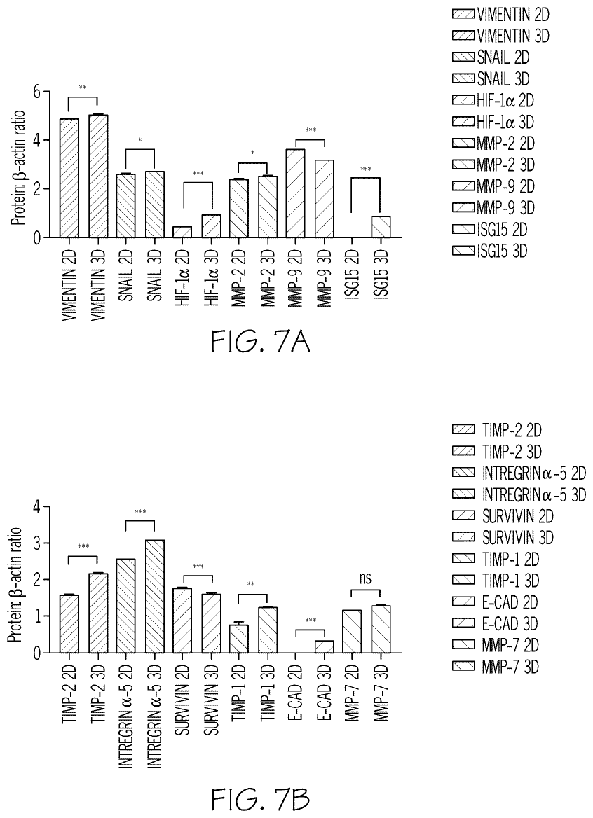

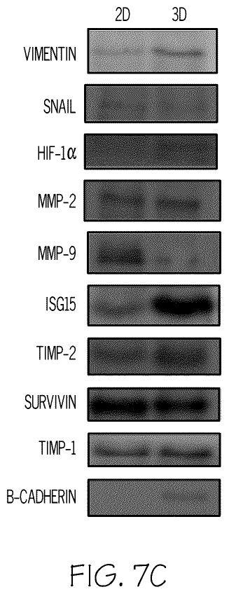

Additionally, AsPC-1 cells were bioprinted using the novel bioink. Protein expression changes were monitored between AsPC-1 monolayer culture and AsPC-1 Bioprinted tumor models after a period of 10 days. FIGS. 7A-7B show quantified protein expression as a ratio of protein:β-actin. FIG. 7C shows representative blots of certain proteins depicting changes. Values were plotted as means ±SD.

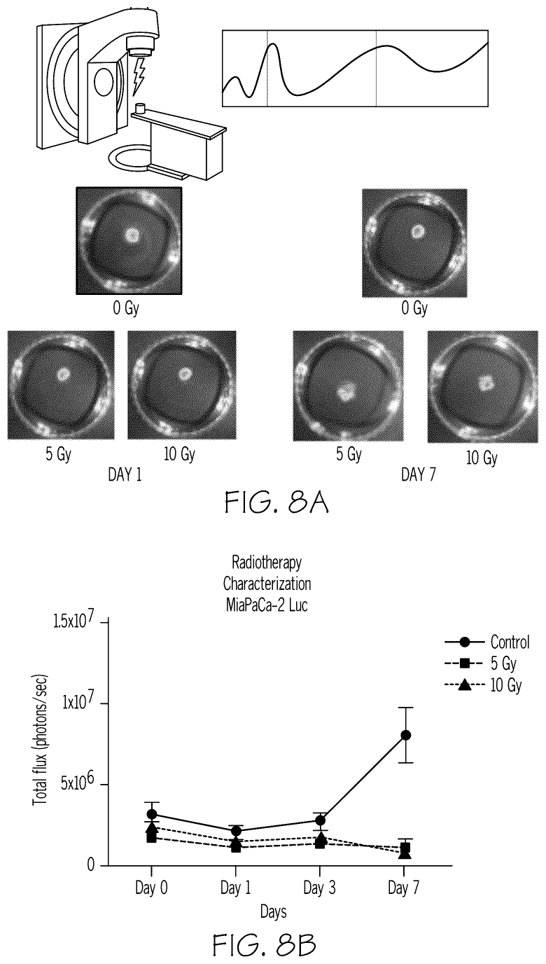

As shown in FIGS. 8A-8B, luciferase expressing MiaPaCa-2 cells were used to create bioprinted tumors. Subsequently, the tumors were divided in three groups: control, 5 Gy and 10 Gy to assess the effect of irradiation on the tumor growth. The luciferase activity was monitored on the day of printing (Day 1) followed by exposure to radiation dose every three days for 1 week. Values were plotted as means ±SD.

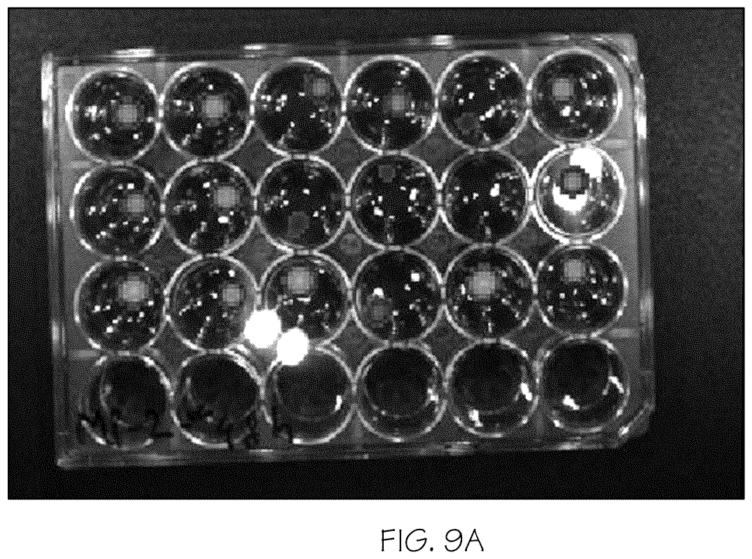

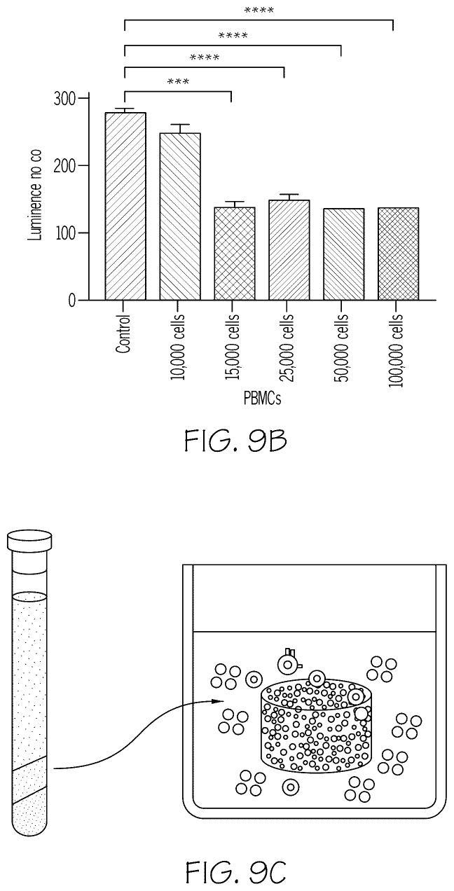

As shown in FIGS. 9A-9C, bioprinted tumors created using luciferase expressing MiaPaCa-2 cells were used to analyze the effect of human PBMCs for their anti-cancer activities. Human PBMCs were isolated from leukocyte reduction chambers (Oklahoma Blood Institute Ltd.). The isolated PBMCs were then added to wells containing bio prints and monitored for one week. Luciferase activity was monitored using IVIS Imaging system. Values were plotted as means ±SD.

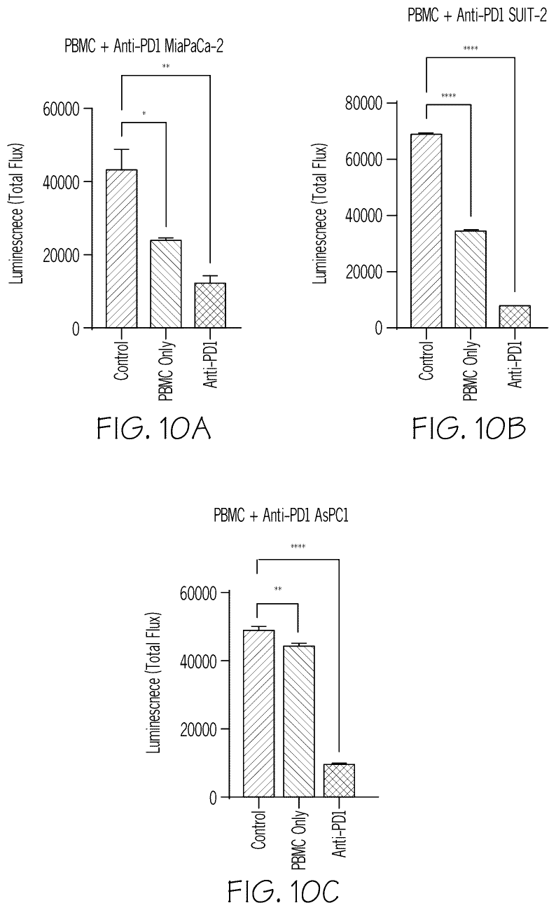

The experimental results in FIGS. 10A-10C further illustrate that bioprinted tumors can serve as a novel platform for immunotherapeutic analysis. Bioprinted tumors containing an immune microenvironment around them were subjected to an immunotherapy called anti-PD1. The immunotherapy increased activity of cytotoxic T cells causing a reduction in the viability of cells within the tumors. The results demonstrate that bioprinted tumors can serve as a reliable method of testing new agents which target the human immune system.

### Example 1.2. Discussion of Experimental Results

Bioprinting is a next generation outcome of the 3D printing technique. For life science research, creating bioprints of the tissue of interest was a difficult task until the advent of extrusion bioprinters. In the current study, Applicant combined extrusion bioprinting and an in house bioink to develop bioprinted PDAC tumors (FIGS. 2A-2B). The bioprinted tumors showed a high viability of 80-93% post-printing. Additionally, the cells displayed an optimal proliferation rate within the bioprints, thus ensuring no toxic effects on cells during the print process.

### Example 1.2.1. Design of Experiments Approach in Bioprinting

Since an in house bioink was utilized to create the tumors, Applicant used the DOE approach (FIGS. 3A-3D and FIGS. 4A-4C). Approximately 24 runs were required to optimize the printing temperature and pressure. Cell viability was used as the output to establish printing parameters and the target viability of 98-100% was considered for an optimized condition. The results affirmed that the bioink is able to support the viability of patient derived cancer cells.

### Example 1.2.2. Bioprinted Tumors Introduce the Natural 3D Phenotype to Cells

Applicant utilized SEM imaging to confirm the shape of cells post-printing. After fixing the bioprinted tumors, the 3D shape of PDAC cells was analyzed and Applicant observed a phenotype closer to an actual in vivo tumor tissue (FIGS. 5A-5C). Moreover, Applicant observed cell-cell interactions as well as cell-matrix interactions. Since these interactions are a critical component of the tumor environment, Applicant's findings support the use of bioprinting for translational studies.

### Example 1.2.3. PDAC Cells Acquire Resistance in a 3D Microenvironment

In order to study the effect of standard chemotherapies on Bioprinted tissues, Applicant analyzed bioprinted PDAC tumors for their response to several anti-cancer drugs (FIGS. 6A-6H). Applicant observed a significant change in the IC₅₀ values between mono-layer culture and 3D tumors. Significant shifts in IC₅₀ values were observed at 24 and 72 hours post treatment with anti-cancer drugs, thus affirming the fact that 3D phenotype confers drug resistance to cancer cells.

### Example 1.2.4. The Natural 3D Environment Confers Significantly Different Protein Expression Patterns

Bioprinted tumors were analyzed for protein expression changes post-printing (FIGS. 7A-7C). As opposed to mono-layer cultures, Applicant observed higher expression of proteins related to invasion, metastasis, hypoxia and some proteins which cause immunosuppression in the TME. The absence, or lower expression of such proteins in the conventional 2D systems makes it difficult to translate the in vitro findings to clinical settings. On the other hand, bioprinted tumors move closer to the real in vivo setting where factors such as invasion and metastasis are present.

### Example 1.2.5. Bioprinted Tumors Serve as a Platform for Radiotherapy Analysis

Radiotherapy has been an important part of the anti-cancer arsenal. However, cancer cells develop resistance against irradiation following chronic exposure. Additionally, the toxic effects of radiation affects the patient's performance status. An in vitro platform to study and optimize radiation dose is a better way to plan treatment regimens. In this Example, Applicant established Bioprinted tumors as a platform to study the effect of irradiation dose on tumors (FIGS. 8A-8B). Applicant observed a significant change in the tumor cell viability at 5 Gy and 10 Gy radiation dose on MiaPaCa-2 luc bioprints.

### Example 1.2.6. Bioprinted Tumors Serve as a Platform for Immunotherapy Analysis

Applicant also established bioprinted PDAC tumors as a platform for immunotherapy studies using human PBMCs (FIGS. 9A-9C). PBMCs were isolated from LRS chambers and were added in different concentrations to the media surrounding bioprints. PBMCs were kept in active state using mitogens such as concanavalin-A. Over a period of 1 week, Applicant observed reduction in the luciferase activity, suggesting a reduced cell viability. Thus, an in vitro artificial immune environment was established.

As illustrated in FIGS. 10A-10C, bioprinted tumors containing an immune microenvironment around them were subjected to an immunotherapy called anti-PD1. The immunotherapy increased activity of cytotoxic T cells causing a reduction in the viability of cells within the tumors. The results demonstrate that bioprinted tumors can serve as a reliable method of testing new agents which target the human immune system.

### Example 1.2.7. Bioprinted Tumor (PDAC) On-Chip Model

As illustrated in FIGS. 1A-1C, Applicant also developed a system for housing the bioprinted PDAC tumors. The system can include Bluetooth-enabled pumps that reduce size of the overall equipment. Small size ensures sterility since the whole equipment can be placed inside an incubator to receive appropriate temperature, humidity and CO₂ levels. Fluid mechanics can be controlled remotely.

Taken Together, Applicant's results demonstrate that in vitro bioprinted tumor models can serve as an optimal in vitro platform to study anti-cancer activity.

### Example 2. Bioprinted Blood Vessels

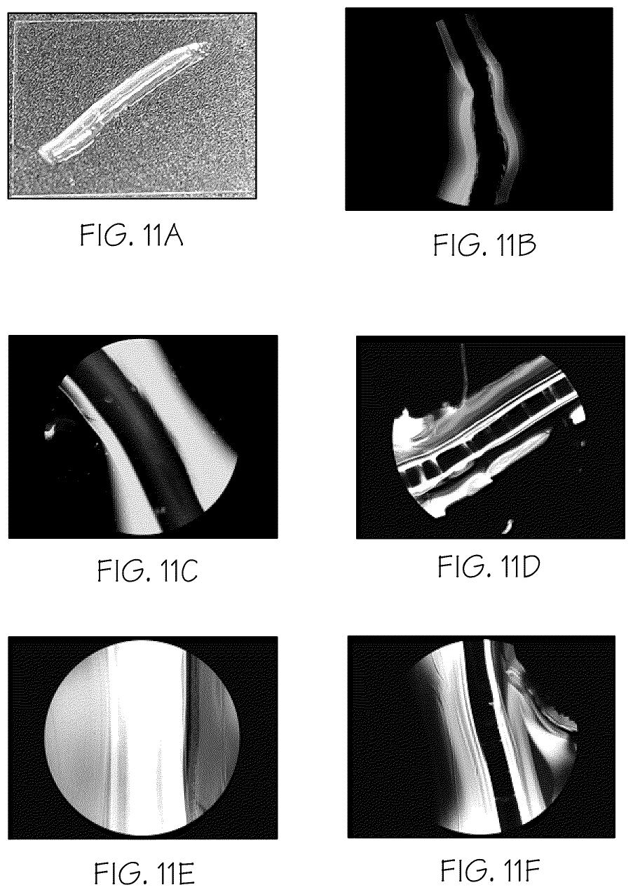

Bioprinting of blood vessels was carried out using co-axial bioprinting wherein two robotic arms of the bioprinter were used. One arm extruded a crosslinking agent while the other arm extruded the bioink made out of sodium alginate, gelatin and cells. The crosslinking agent was flown at a high pressure to create a hollow tube-like space through the bioink. This led to creation of the hollow tubing which mimics the vasculature part of a tissue. FIGS. 11A-11F show the structure of the vessel. FIGS. 11B, 11C and 11F contain a blue colored dye to confirm formation of the hollow cylindrical tube-like space post-printing.

### Example 3. Evaluation of Tumor Stiffness

The bioprinted tumor described in Example 1 was characterized for its stiffness since tumor stiffness affects the efficacy of anti-cancer therapies. In particular, the bioprinted tumor was subjected to elastography measurements where the stiffness was measured in Young's modulus (kPa). The results are summarized in Table 1.

TABLE 1
Tumor stiffness measurements.

| B Mode | Young's Modulus |
|---|---|
| Measurements | Value (kPa) |
| 1 | 6.12 |
| 2 | 6.85 |
| 3 | 13.2 |
| 4 | 16.69 |
| 5 | 25.60 |
| 6 | 32.66 |
| 7 | 42.67 |
| 8 | 47.5 |
| 9 | 45.98 |
| 10 | 51 |

As summarized in Table 1, 10 different measurements of the tumors were taken. Measurements number 1 and 2 are without cells. The other measurements are with cells incorporated in the bioink. The results indicate that the tumor showed stiffness similar to what is observed in actual human tumors from clinical samples.

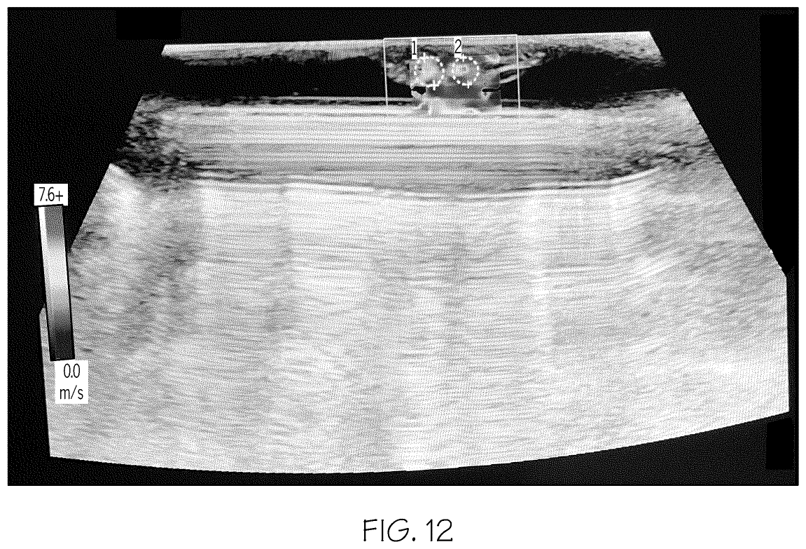

FIG. 12 shows an actual elastography of the bioprinted tumor. The measurement was performed on GE Logiq E9.

### Example 4. Bio-Printed Bone-on-Chip

This Example describes a three-dimensional bio-printed bone-on-chip structure that resembles bone tissue and bone architecture, such as the human bone structure. As illustrated in FIG. 1B, this structure contains natural, non-synthetic materials, such as osteoblasts, bone cancer cells, osteoclasts, bone marrow cells, hydroxyapatite bone materials, and connective tissue to mimic bone tissue. The structure further contains vasculature channels to allow the flow of fluids (e.g., blood) through the bone. The model can also include a central channel and added fibrin. This technology enables simulations of interactions within the bone microenvironment and may be used as a model for the in vitro study of bone-related diseases.

The three-dimensional bio-printed bone-on-chip structure can be embedded into a chip-platform, which provides channels and vascularization for media to flow through the organoid, as well as into other vessel chambers. For instance, the structure can include Bluetooth pump controlled vasculature channels, thereby allowing better user interface and reduced size of the product. This enables the simulation of physiological conditions and interactions within the bone microenvironment. The model can be used to study various bone-related diseases, such as osteoarthritis, osteocarcinoma, bone-related cancers, bone cancer metastasis, or combinations thereof.

For instance, the model can include certain differentiators, such as lung tissue and bone tumor, which are metastatic sites for bone cancer cells. As such, the model can be used to study metastasis. For instance, the model can also include lung tissue, thereby allowing for the study of bone cancer metastasis.

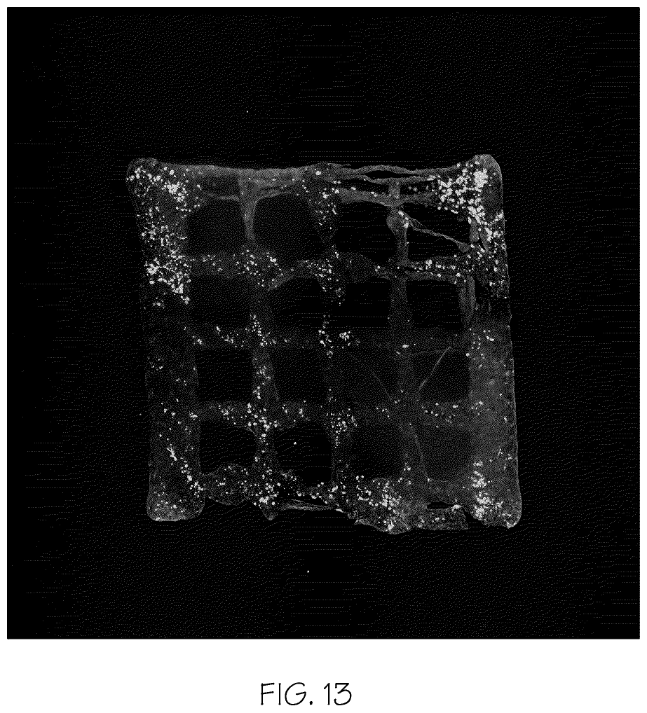

The results of a cell viability assay of a bone tumor model are shown in FIG. 13. The cells embedded in the bioprinted bone are stained with calcein AM and ethidium homodimer. Calcein AM stains the live cells while ethidium homodimer stains the dead cells. Both the dyes are added together and the cells are stained for at least 25 minutes. After incubation, the bone is washed thrice with PBS and observed under a confocal microscope. Cells stained in green are live cells while the red colored staining confirm cell death. Based on the proportion of green and red fluorescence, the number of live and dead cells can be evaluated to understand viability of the bone tumors. The results confirmed that the bone tumors are viable for at least 30-40 days. Such prolonged viability provides ample time for investigators to study the bone model, such as through compound testing.

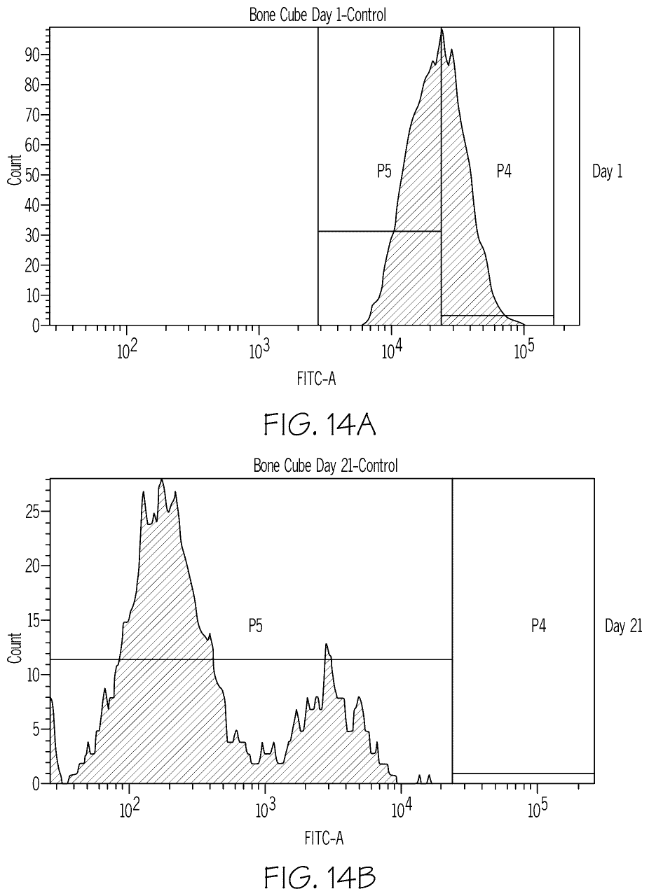

The results of a cell proliferation experiment of the bone tumor model are shown in FIGS. 14A-14B. To confirm proliferation, the cells are stained with CFSE dye prior to addition in the bone scaffold. Once the stained cells get embedded in the scaffold, the intensity of the dye is monitored using flow cytometry. The intensity of CFSE dye in the bone tissue reduces as the cells proliferate. Movement of peaks towards the left in FIG. 14B represent the proliferation of mother cells into daughter generations. The results confirmed that the cells proliferate to next generations, thus mimicking bone tumor growth.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A bioink operational to form an artificial bone, wherein the bioink comprises
a plurality of bone-related cells.

2. The bioink of claim 1, wherein the plurality of bone-related cells are selected from the group consisting of bone tissue cells, lung tissue, tumor cells, cancer cells, bone cancer cells, immune cells, fibroblasts, osteoblasts, osteoclasts, bone marrow cells, or combinations thereof.

3. The bioink of claim 1, wherein the plurality of bone-related cells are selected from the group consisting of bone cancer cells, osteoblasts, osteoclasts, bone marrow cells, or combinations thereof.

4. The bioink of claim 1, wherein the bioink further comprises one or more components selected from the group consisting of sodium alginate, gelatin, fibronectin, laminin, hydroxyapatite bone materials, fibrin, poly-caprolactone, osteocalcin, osteonectin, ostopontin, or combinations thereof.

5. The bioink of claim 1, wherein the bioink further comprises one or more components selected from the group consisting of hydroxyapatite bone materials, fibrin, or combinations thereof.

6. The bioink of claim 1, wherein the bioink further comprises collagen.

7. The bioink of claim 6, wherein the bioink comprises at least 20% by weight of collagen.

8. The bioink of claim 6, wherein the collagen comprises at least collagen I, collagen III, and collagen IV.

9. The bioink of claim 6, wherein the bioink comprises at least 25% by weight of collagen I, at least 5% by weight of collagen III, and at least 1% by weight of collagen IV.

10. An artificial bone, wherein the artificial bone comprises:
a plurality of bone-related cells; and
a bioink embedded with the cells,
wherein the artificial bone is in the form of a three-dimensional structure comprising a plurality of channels.

11. The artificial bone of claim 10, wherein the plurality of channels are operational to allow fluid flow through the artificial bone.

12. The artificial bone of claim 10, wherein the plurality of channels comprise at least one channel extending through an entire length of the artificial bone.

13. The artificial bone of claim 10, wherein the artificial bone is in the form of a bone cancer model.

14. The artificial bone of claim 10, wherein the plurality of bone-related cells are selected from the group consisting of bone tissue cells, lung tissue, tumor cells, cancer cells, bone cancer cells, immune cells, fibroblasts, osteoblasts, osteoclasts, bone marrow cells, or combinations thereof.

15. The artificial bone of claim 10, wherein the plurality of bone-related cells are selected from the group consisting of bone cancer cells, osteoblasts, osteoclasts, bone marrow cells, or combinations thereof.

16. The artificial bone of claim 10, wherein the bioink further comprises one or more components selected from the group consisting of sodium alginate, gelatin, fibronectin, laminin, hydroxyapatite bone materials, fibrin, poly-caprolactone, osteocalcin, osteonectin, ostopontin, or combinations thereof.

17. The artificial bone of claim 10, wherein the bioink further comprises one or more components selected from the group consisting of hydroxyapatite bone materials, fibrin, or combinations thereof.

18. The artificial bone of claim 10, wherein the bioink further comprises collagen.

19. The artificial bone of claim 18, wherein the bioink comprises at least 20% by weight of collagen

20. The artificial bone of claim 18, wherein the collagen comprises at least collagen I, collagen III, and collagen IV.

21. The artificial bone of claim 20, wherein the bioink comprises at least 25% by weight of collagen I, at least 5% by weight of collagen III, and at least 1% by weight of collagen IV.

22. The artificial bone of claim 10, wherein the artificial bone is associated with a bone maintenance system comprising:
a housing unit for housing the artificial bone; and
a nutrient source in fluid communication with the housing unit.

23. The artificial bone of claim 22, wherein the bone maintenance system further comprises one or more additional housing units in fluid communication with the housing unit, wherein the one or more additional housing units contain one or more additional tissues.

24. A method of making an artificial bone, said method comprising:
applying a plurality of bone-related cells and a bioink onto a surface,
wherein the bioink becomes embedded with the cells, and
wherein the formed artificial bone is in the form of a three-dimensional structure comprising a plurality of channels.

25. The method of claim 24, wherein the applying occurs by three-dimensional printing.

26. The method of claim 24, wherein the formed artificial bone is utilized for screening therapeutics, wherein the therapeutics comprise radiation therapy, chemotherapy, immunotherapy, drugs, drug candidates, or combinations thereof.

27. The method of claim 24, wherein the formed artificial bone is utilized for evaluating one or more bone-related diseases selected from the group consisting of osteoarthritis, osteocarcinoma, bone-related cancers, bone cancer metastasis, or combinations thereof.

28. The method of claim 24, wherein the plurality of bone-related cells are selected from the group consisting of bone tissue cells, lung tissue, tumor cells, cancer cells, bone cancer cells, immune cells, fibroblasts, osteoblasts, osteoclasts, bone marrow cells, or combinations thereof.

29. The method of claim 24, wherein the bioink further comprises one or more components selected from the group consisting of sodium alginate, gelatin, fibronectin, laminin, hydroxyapatite bone materials, fibrin, poly-caprolactone, osteocalcin, osteonectin, ostopontin, or combinations thereof.

30. The method of claim 24, wherein the bioink further comprises collagen.

31. The method of claim 30, wherein the bioink comprises at least 20% by weight of collagen

32. The method of claim 30, wherein the collagen comprises at least collagen I, collagen III, and collagen IV.

33. The method of claim 30, wherein the bioink comprises at least 25% by weight of collagen I, at least 5% by weight of collagen III, and at least 1% by weight of collagen IV.

34. The method of claim 24, wherein the bioink and the bone-related cells are applied onto the surface at the same time.

35. The method of claim 24, wherein the bioink and the bone-related cells are applied onto the surface sequentially.

36. The method of claim 35, wherein the method comprises:
applying the bioink onto the surface to form the three-dimensional structure comprising the plurality of channels; and
applying the bone-related cells onto the three-dimensional structure.

37. The method of claim 36, wherein the bone-related cells are applied into the plurality of channels of the three-dimensional structure.
